(19) United States
(12) Patent Application Publication
(10) Pub. No.: US 2010/0049027 A1
(43) Pub. Date: Feb. 25, 2010

(54) ELECTRODE BELT FOR CARRYING OUT ELECTRODIAGNOSTIC PROCEDURES ON THE HUMAN BODY

(75) Inventors: Eckhard Teschner, Hamburg (DE); Thomas Gallus, Ratekau (DE); Jianhua Li, Lubeck (DE); Arndt Poecher, Bad Schwartau (DE); Eckhard Riggert, Ratekau (DE); Yvo Garber, Lubeok (DE)

Correspondence Address: McGLEW AND TUTTLE;John James McGlew, Scarborough Station, Scarborough, NY, 10510-9227, US

(73) Assignee: Drager Medical AG & Co. KG

(21) Appl. No.: 12/608,362

(22) Filed: Oct. 29, 2009

Related U.S. Application Data
(63) Continuation of application No. 11/244,114, filed on Oct. 5, 2005.

(30) Foreign Application Priority Data
Oct. 20, 2004 (DE) 10 2004 050 981.6

(51) Int. Cl. A61B 5/04

(52) U.S. Cl. 600/390

(57) ABSTRACT

An electrode belt is provided for carrying out electrodiagnostic procedures on the human body. The electrode belt includes a belt (2), which is formed at least partially of an elastic material and surrounds the body of a test subject. A plurality of electrodes, are mechanically connected to the belt and are in flat contact with the body of the test subject. At least one contact passes from the electrodes through the belt (2) and is connected to a connection element (11), which is connected to a lead each of a multicore cable (6). Such an electrode belt offers marked advantages in terms of handling along with increased safety from movement artifacts.

### CROSS REFERENCE TO RELATED APPLICATIONS

This is a continuation under 37 CFR 1.53(b) of pending prior application Ser. No. 11/244,114 filed Oct. 5, 2005 and claims the benefit of priority under 35 U.S.C. §119 of German Application DE 10 2004 050 981.6 filed Oct. 20, 2004. The entire contents of each of the applications is incorporated herein by reference.

### FIELD OF THE INVENTION

The present invention pertains to an electrode belt for carrying out electrodiagnostic procedures on the human body.

### BACKGROUND OF THE INVENTION

It has been known for a long time that electric signals, which are obtained via electrodes applied to the body, can be evaluated in connection with various diagnostic procedures. The application of electrodes on the body surface, which is necessary for this, must guarantee primarily reliability and stability of position. Large contact areas are used, in general, for obtaining signals in a reliable manner in order to ensure good electric contact.

Two basic principles have become widespread, in principle, for attaching the electrodes. Electrodes are either attached to the body surface as individually adhering electrodes, or the electrodes are attached to a carrier means, which ensures the reliable seating of the electrodes in the desired positions. Numerous different carrier means have been known for such an attaching of electrodes. General requirements, which are also to be imposed on other devices that are intended for use in the field of medicine, are imposed, as a rule, on these [carrier means]. These may include a design as a reusable product and, associated herewith, good suitability for easy cleaning or disinfection or sterilization. Furthermore, low production costs are always sought to be achieved: Besides, the possibility of rapidly arranging such a carrier means even on recumbent or unconscious patients, which must possibly be possible by a single care person, is to be provided.

Furthermore, requirements that ensure the acceptance of the particular carrier means and of the diagnostic procedure that can be embodied therewith are to be taken into consideration. Forms of geometric design, i.e., for example, a flat design, which limit the particular patient's mobility only slightly at best, are therefore desired. A stretching behavior, which can be metered in a pleasant manner and is not felt to be disturbing, a surface quality that does not lead to discomfort on the part of the patient, as well as a pleasant wear behavior even in case of long-term applications are desirable in case of elastic devices. Particular attention should be paid, besides, to efforts to find embodiments that lead to a minimal formation of necroses at best during long-term applications.

Various forms of belts have proved to be particularly successful as carrier means for diagnostic procedures in which electrodes must be arranged essentially in one plane around a patient's body or only individual electrodes must be attached to the body.

The electroimpedance tomography procedure is such a procedure, which requires the arrangement of a plurality of electrodes essentially in one plane around the body of a patient. Electroimpedance tomography is a procedure in which the electric alternating current impedance between the feed point and the measurement point can be calculated by feeding an electric alternating current into the human body and measuring the resulting surface potentials at different points of the body. A two-dimensional tomogram of the electric impedance distribution in the body being examined can be determined by means of suitable mathematical reconstruction algorithms with different combinations of the feed site and measurement site, for example, by successive rotation of the position of the current feed around the body while measuring the surface potentials at the same time along a section plane.

A tomogram of the impedance distribution of the human body is of interest in medicine, because the electric impedance changes with both the air content and the content of extracellular fluids in the tissue. Thus, both ventilation, especially the distribution of air-filled cavities, and perfusion can be visualized and monitored within the section plane in a regionally resolved manner. This is of significance especially in examinations of the thorax.

Since electroimpedance tomography imposes relatively high requirements on the application of the electrodes, the present invention shall be explained below as an example on the basis of this procedure even though the technical teaching can be extrapolated without problems to other electrodiagnostic procedures as well.

The reliable and rapid connection of the electrodes to recumbent patients as well as a permanently good contact between the skin and the individual electrodes are of crucial significance for the clinical acceptance of the electroimpedance tomography procedure. The use of standard electrodes, i.e., for example, commercially available ECG electrodes, belongs to the state of the art. These are frequently connected to individually extending electrode cables. To suppress electric interferences and strong inductive disturbance, these electrodes are connected to the electroimpedance tomography apparatus in some cases via shielded lines. This shielding can be operated actively in some cases. Since electroimpedance tomography is a procedure in which signals fed in are needed that must be known accurately in order to make possible the meaningful evaluation of received signals, this procedure inherently has an especially high susceptibility to coupled interferences.

Various processes have been known for reducing such interferences by means of specific filter algorithms or for minimizing them by corresponding calibrations concerning their effect. However, since individual cables may also interfere with one another, a relative stability of the positions of the individual cables in relation to one another is absolutely necessary for such a calibration. This requirement can be met for a large number of cables at considerable effort only. A larger number of individually extending cables is, moreover, less comfortable for the patient as well as the medical staff. To guarantee low susceptibility to errors, overstressing of these lines is to be avoided in connection with the use of electric lines. In particular, kinking and strong tensile loads are to be avoided.

Numerous approaches to partially master these problems have been known from the state of the art.

It is known from a device of this class that a plurality of electrodes can be arranged at a support structure and they can be actuated and polled through individual lines, which lead to a multipole cable. However, this device is suitable preferably for performing ECG examinations. This device is susceptible to interferences in case of application in the area of electroimpedance tomography, because the individual lines lack sufficiently stable positions (WO 97/14346).

Furthermore, it is known that a plurality of electrodes can be cast in one piece with a belt. However, this sometimes causes the manufacturer to face considerable difficulties and reduces the subsequent possibility of adaptability of such a belt system to special requirements (WO 03/082103 A1).

Furthermore, it is known that a plurality of electrodes can be arranged on an elastic band. However, such a solution possibly offers an excessively low level of safety concerning the stability of position of the electrodes under various conditions of use (DE 196 10 246 A1).

Furthermore, it is known that the susceptibility to interferences of a described device with a plurality of electrodes can be reduced by special driver circuits. However, this causes a rather substantial increase in the technical effort and offers only conditional safety against the effects of various coupled interferences (DE 101 56 833 A1).

Furthermore, it is known that the meaningfulness of images obtained by electroimpedance tomography can be increased by superimposing to these images other images that were obtained by other physical diagnostic procedures. However, this considerably increases the technologically necessary effort (EP 1000580 A1).

### SUMMARY OF THE INVENTION

The object of the present invention is to provide an electrode belt that extensively avoids the above-described drawbacks of the state of the art and is, in particular, well suited for use in the area of electroimpedance tomography.

The present invention is embodied by an electrode belt for carrying out electrodiagnostic procedures on the human body. This electrode belt comprises a belt, which consists at least partially of an elastic material and surrounds the body of a test subject, and a plurality of electrodes, which are mechanically connected to the belt and are in flat contact with the body of a test subject, wherein at least one contact means passes through the belt from the electrodes and is connected to a connection element, which is connected to a respective lead of a multicore cable. The contact means is designed for this purpose as a conductive connection between the electrode surface and the connection element, it is firmly connected in an advantageous embodiment to the electrode and has a sufficient thickness to hold, as a support means, the connection element in its position.

It is advantageous if each lead of the multicore cable has a separate shielding.

The embodiment in which the leads are led within a multicore cable guarantees nearly constant position of the individual leads in relation to one another. The effects of different interferences can be effectively reduced in connection with the separate shielding of each lead. Such a multicore cable may be defined as a cable tree comprising a plurality of individually shielded cables, which is characterized by especially good positional stability of the individual cables in relation to one another and by especially easy handling.

An advantageous applicability of an electrode belt according to the present invention can be embodied if the contact means connected with the electrodes are detachably connected to the connection elements, which are connected to a lead each of the multicore cable. The complete cable structure including the connection elements can thus be separated from the electrode belt without having to remove the electrodes from the support structure of the belt. It is particularly advantageous for such an embodiment if each electrode has a rigid contact pin each, which is passed through the belt and protrudes from the belt on the side of the belt facing away from the body. This contact pin is preferably connected detachably with a corresponding connection element. The connection is advantageously carried out in the manner of a pushbutton connection. For example, the contact pins of the electrodes may have for this purpose a spherical closure on the side facing away from the body. The connection elements, which are connected to a lead each of the multicore cable, have, in their turn, spring elements, which make it possible to reach behind the spherical closure of the contact pins. The pushbutton connection can thus be brought about by simply pressing the connection elements on the contact pin and pulling them off the contact pin, or it can be supported by unlocking aids contained in the connection elements. For example, cable systems as described in US 2004/0105245 A1 may be used for this embodiment. Highly reliable results were thus obtained in a signal feed frequency range of 50-200 kHz. Very low interference levels can be reached by means of a cabling arranged in this manner with separate shielding of the individual leads. The inductive disturbance between the leads is, moreover, well compensated; handling is very user-friendly, and movement artifacts due to changes in the distance between individual leads are reduced as well. The possibility of separating the belt and the cable from one another is advantageous for cleaning operations which become necessary in the course of everyday use.

It is particularly advantageous for increasing wearing comfort if the electrodes have a planar surface, which is in flat contact with the test subject's body and the edge of the electrodes ends approximately flush with the belt surface lying on the body. An especially high reliability of contact is obtained if the electrodes have a convex surface, which is in flat contact with the test subject's body and the edge of the electrodes ends approximately flush with the belt surface lying on the body. Due to the elevation of the convex surface, there will be an especially close contact between the electrode and the surface of the body at least in the middle area of the electrode surface.

Moreover, it is advantageous especially for long-term applications if only mild skin irritations occur at best at the edges of the belt. One problem arises due to the fact that such electrode belts are frequently used in relatively obese patients. The pressing pressure of the belt that is necessary for a reliable electric contact may possibly cause the belt to cut relatively deeply into the skin. To nevertheless prevent skin irritations at the edges of the belt even during long-term wear, it is advantageous if the thickness of the belt material decreases toward the edges. Easier deformability of the edges of the belt is achieved as a result, which prevents the skin from overlapping in this area because the belt can adapt itself better to the shape of the skin and a sharp-edged termination cannot occur. Skin irritations in the edge area of the electrode belt can be prevented from occurring especially effectively if the edges of the belt are designed as a hose-like bead. As a result, the skin cannot form folds, which would have edges that would touch each other, even if the belt penetrates relatively deeply into the patient's skin, but it can gather only around areas of the hose-like bead. It was found that such an embodiment of the belt contributes to the effective prevention of necroses even during long-term applications.

To guarantee protection of the cables from excessive pull, it is advantageous if the distance between adjacent electrodes in the relaxed state is shorter than the length of the multicore cable between the corresponding adjacent connection elements and the multicore cable has a meandering course extending approximately in parallel to the body surface. It proved to be especially advantageous if the cable is approx. 30% longer than the belt in the relaxed state. If the elastic belt is stretched, only the length of the belt will change, but the length of the cable will remain constant, and the meander structure, in which the cable is led, will be flattened, instead. Thus, in case of a 30% longer cable, stretching of the belt by 30% can be achieved without the tensile load on the cable changing. This acts as a securing against overload when the belt is stretched to the full length of the cable. It is especially advantageous for a meandering cable pattern if the connection elements are connected to the contact means of the electrodes such that they can be rotated about an axis in parallel to the normal line to the body surface in the area of the flat contact of the electrodes. This makes it possible to firmly clamp the cable in the connection elements. Kinking of the cables under different tensile loads is thus completely prevented from occurring. Long-term stable use without a necessary change of cable is thus ensured. The quality of the signals is essentially maintained because sensitive kinks are prevented from forming in the cable.

It is especially advantageous if the belt and/or the electrodes consist of a material that can be disinfected and sterilized without being damaged. Silicone as the belt and/or electrode material is provided for this purpose in an advantageous embodiment. Moreover, it is advantageous for maintaining the contact properties if the electrodes consist at least partially of a conductive plastic or a plastic coated with a conductive material. In an alternative advantageous embodiment, the electrodes consist partially of stainless steel or sintered silver chloride.

An especially high reliability of the electrode contact and electrode position is obtained if at least individual electrodes have adhesive gel pads on one side. To facilitate the placement of an electrode belt according to the present invention, it is advantageous if the electrode belt can be opened at least at one point. It is especially advantageous if the belt comprises a plurality of individual segments that can be connected with one another and each of these individual segments is connected with at least four electrodes. These four electrodes each are advantageously connected to connection means that are connected to different, individually shielded leads of a multicore cable each. The individual segments with the electrodes and the cable can thus be separated from one another in the completely mounted state and can be individually replaced or put on one after another.

It is advantageous in connection with the use of the electrode belt according to the present invention in the area of the chest if tensioning means, which ensure the firm seating of individual electrodes in concave areas of the body surface, are additionally present. These tensioning means may be designed such that at least one gel pad is present, which makes it possible to support the electrodes at a tensioning means arranged at a spaced location in front of the body, for example, at a belt.

An especially effective shielding against interferences can be achieved if the individual leads of the multicore cable are doubly shielded. In another, especially effective embodiment, the individual leads of the multicore cable have an individual shielding each and are additionally surrounded as a whole by a second, common shielding. Individual shieldings or all shieldings may be driven actively or act passively.

An especially high reliability of operation can be achieved if the connection between the belt and the connection elements is designed such that liquids are prevented from penetrating into the area in which the electric contact is established or the penetration of liquids is at least made difficult. This can be achieved, for example, by molding seals on the belt, which engage a groove in the body of the individual connection elements in a positive-locking manner.

The present invention will be explained in greater detail on the basis of an exemplary embodiment.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a sectional view of an electrode belt according to the present invention with electrodes with a planar contact surface;

FIG. 2 is a sectional view of an electrode belt according to the present invention with electrodes with a convex contact surface;

FIG. 3 is a schematic overall view of an electrode belt according to the present invention;

FIG. 4 is the view of an electrode belt according to the present invention in the relaxed state;

FIG. 5 is the view of an electrode belt according to the present invention in the tensioned state;

FIG. 6 is a sectional view of an especially advantageous belt shape;

FIG. 7 is a schematic view of an electrode belt according to the present invention with a gel pad for supporting the electrodes in the area of the sternum;

FIG. 8 is a schematic view of a connection element with an electrode attached thereto; and

FIG. 9 is a schematic view of a connection element with an electrode attached thereto, wherein the area of the electric contact is secured against the penetration of liquids.

### DESCRIPTION OF THE PREFERRED EMBODIMENTS

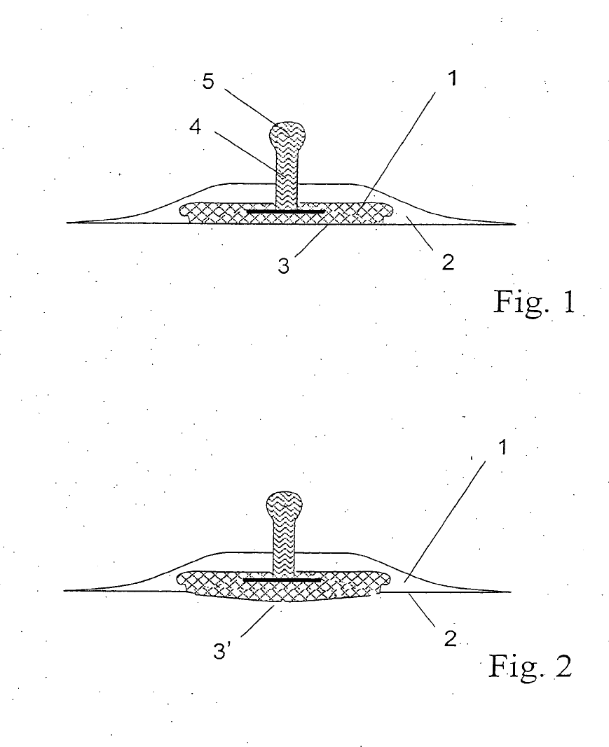

Referring to the drawings in particular, FIG. 1 shows, in a sectional view of an electrode belt according to the present invention, how an electrode 1 is integrated in an elastic belt 2. The electrode 1 has a planar contact surface 3. This ends flush with the belt 2, so that a uniform, flat surface is formed on the patient's body. A contact pin 4 passes through the belt 2, protrudes from the belt 2 on the side facing away from the body, and has a spherical closure 5. This spherical closure 5 can be introduced into a corresponding connection element according to the pushbutton principle. Attaching of the connection elements, in which the connection elements are mounted rotatably about an axis at right angles to the electrode surface, even though they are fixed in a position, can be achieved by means of this pushbutton connection in an especially simple manner. The thickness of the belt material decreases from the center toward the edge, which leads to increased wear comfort. As a whole, this embodiment makes possible a very flat design.

FIG. 2 shows a similar design according to the present invention with an electrode 1, which has a convex contact surface 3′. As a result, the contact surface 3′ slightly projects from the belt 2, which ensures an especially effective contact with the patient's skin. In addition, the entire contact surface 3′ projects slightly over the belt material. This projection is dimensioned such that the skin will not be damaged and there will be no loss of wear comfort. The edge areas of the belt 2, which end flat, ensure even in case of highly obese patients and relatively strong pressing pressures that there will be no skin irritation at the edge of the belt when the belt cuts into the skin. The flatly tapering areas of the belt possibly fit the shape of any possible skin fold.

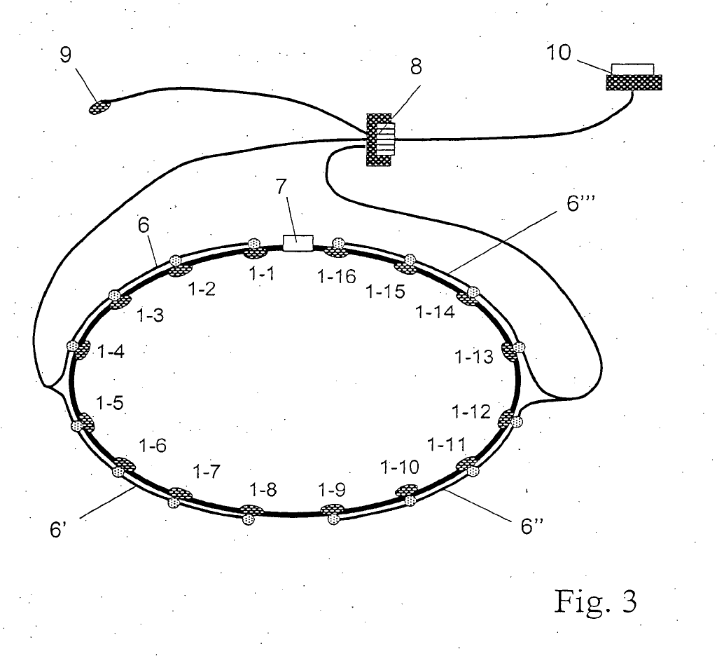

FIG. 3 shows a schematic overall view of an electrode belt according to the present invention, which surrounds a patient's upper body. This comprises a belt 2 made of a stretchable biocompatible material, such as silicone, which has a good stretching behavior in case of a slight increase in force and represents hardly any allergenic burden. The electrode belt also comprises in this case 16 firmly integrated electrodes 1-1 through 1-16 made of silicone, which are connected to a cable tree by means of a pushbutton connection on the rear side of the belt. This cable tree comprises essentially one or more multicore cables, whose individual leads are shielded individually. In this exemplary embodiment, the device contains two electrode groups with eight electrodes each, which are supplied with corresponding cable connections from two directions, wherein four electrodes each are connected via connection elements to a respective common cable 6, 6′, 6″, 6′″. The electrode belt may be advantageously separated at a connection element 7 at intermediate points between the electrode groups, here between the electrodes 1-1 and 1-16, in order to facilitate the application. Besides the fixed integration of the electrodes into the belt material, a detachable connection is possible between the electrodes and the belt in another advantageous embodiment, for example, by plugging the electrodes into a belt provided with prepared openings. As a result, especially simple cleaning and disinfection can be achieved and the entire electrode belt can be adapted to changed requirements. Due to the elasticity of the belt material, a pressure that depends on the circumference of the thorax and the length of the belt is applied to the electrodes. The four multicore cables 6, 6′, 6″, 6′″ are led in pairs, on the side of the patient, to a plug type connection 8 located near the patient, to which a reference electrode 9 and a connection cable 10 for connection to an electrode belt are connected.

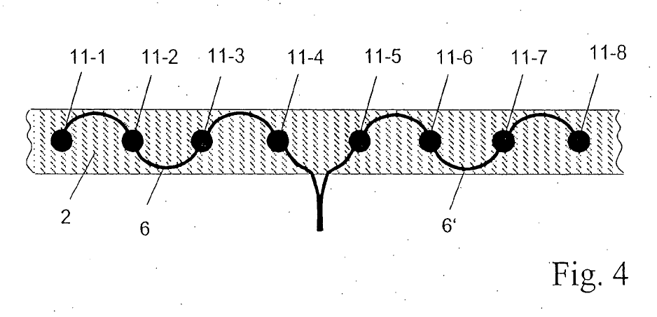

FIG. 4 shows the side of a half of an electrode belt shown in FIG. 3, which side faces away from the body. The eight electrodes are supplied by two multicore cables 6, 6′, four electrodes each being connected via connection elements 11-1 through 11-4 and 11-5 through 11-8 to one and the same multicore cable and the connection elements being each connected to another, individually shielded lead. The belt 2 is in the relaxed state. The length of the multicore cable is approximately 30% greater than the length of the carrying belt 2 in the relaxed state. The connection elements 11-1 through 11-8 are mounted rotatably and have an orientation that enables the multicore cables to have a kink-free, meandering course.

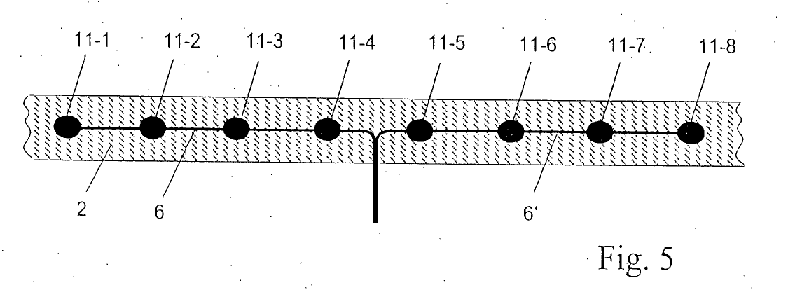

FIG. 5 shows the same detail of an electrode belt according to the present invention in the state in which it is overstretched by 30%. The belt 2 and the multicore cable 6, 6′ are approximately parallel in this situation. The rotatably mounted connection elements 11-1 through 11-8 are pivoted into a position that makes possible the kink-free, parallel course of the cable in front of the belt. In addition, a strain relief integrated in the multicore cable becomes effective in case of this overstretching. A further stretching is not possible, because the multicore cable connected to the belt via the connection elements and the electrodes acts as a stop.

The use of a multicore cable, in which each lead is shielded individually, offers technological advantages. Such cables can be manufactured as cut goods and are cut at the particular necessary points only in case of applications to an electrode belt according to the present invention, and only the lead that is to be connected to the corresponding connection element is actually cut electrically in case of such a cutting, while the other remaining leads extend past the connection point without damage to the shielding or the core. Thus, all leads of the multicore cable extend through the entire length of the multicore cable, and each lead is interrupted once at a different point. This configuration additionally offers more possibilities for an actively driven or passive shielding. The individual shieldings around the leads can additionally be combined with other variants of shielding.

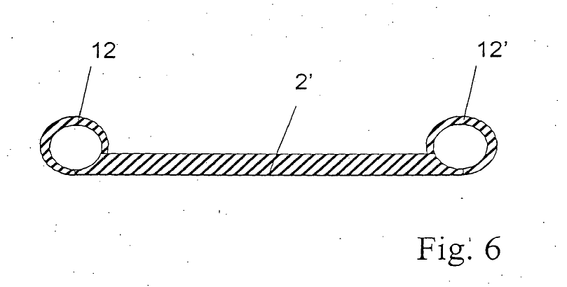

FIG. 6 shows another embodiment of an electrode belt according to the present invention in a schematic sectional view of the carrying belt 2′ without electrodes. The edge areas of this belt are designed as a hose-like bead 12, 12′. This bead prevents sharp skin folds from forming and thus effectively counteracts skin irritations or necroses, even in case of long-term application. In an especially advantageous embodiment, these hose-like beads 12, 12′ may be additionally filled with a gas, for example, air, which makes it possible to set the diameter of the hose-like beads. The carrying properties of electrode belts according to the present invention can thus be adapted in terms of their wear comfort to the requirements of different patients. The embodiment as an elastic round bead without a cavity or the possibility of filling is additionally provided in a simplified form.

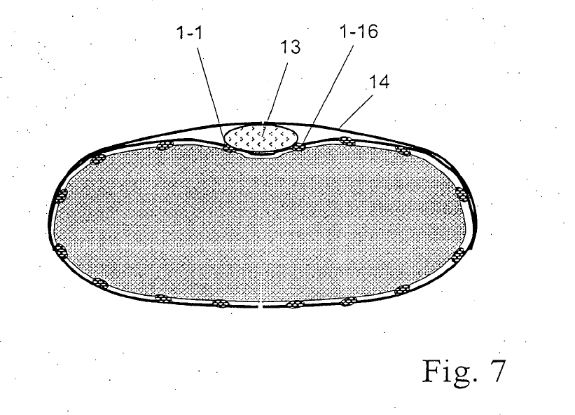

FIG. 7 shows a schematic view of an electrode belt according to the present invention with a gel pad 13 for supporting the electrodes in the area of the sternum. The electrode belt surrounds the entire upper body of a patient. In the area of the sternum, the upper body has a concave area, in which the electrodes have no contact with the skin without auxiliary means with the belt tightened tightly. A belt-like support means 14 is present for this reason, which spans over the concave area of the upper body. The gel pad 13, which is a flexible spacer, can be supported on this. As a result, the necessary pressing pressure can be applied to the electrodes 1-1 and 1-16 via the gel pad 13 in the concave area of the upper body.

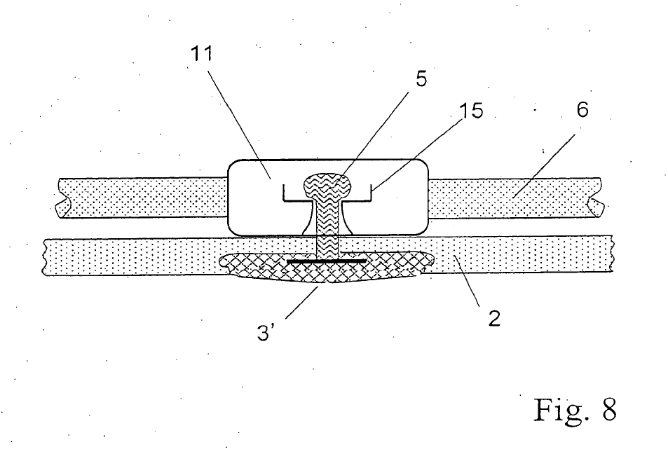

FIG. 8 shows a schematic view of a connection element 11 and an electrode attached thereto with a convex contact surface 3′. The electrode is embedded in an elastic belt 2. The connection element 11 is connected to a multicore cable 6. The individual wires may be connected with each connection element 11 by soldering or by crimping. Spring elements 15, which extend behind the spherical closure 5 of the contact pin and thus ensure a pushbutton-like connection between the electrode 1 and the connection element 11, are arranged inside the connection element. Due to being able to slide around the contact pin, the spring elements 15 make possible the rotatable mounting of the connection element 11, the rotation taking place essentially about the main axis of the contact pin. The spring contact 15 may also be provided formed of an electrically conducting synthetic material. With this, an electrical and mechanical connection may be provided with the connection element 11 to the individual wires by welding or by a melting process. The spring elements 15 may also be provided formed of silicone. The individual wires are then connected with connection element 11 by a vulcanizing process. The spring elements 15 may also be provided formed of various other materials. In such cases the individual wires may be connected with the connection element 11 by means of an electrically conductive glue.

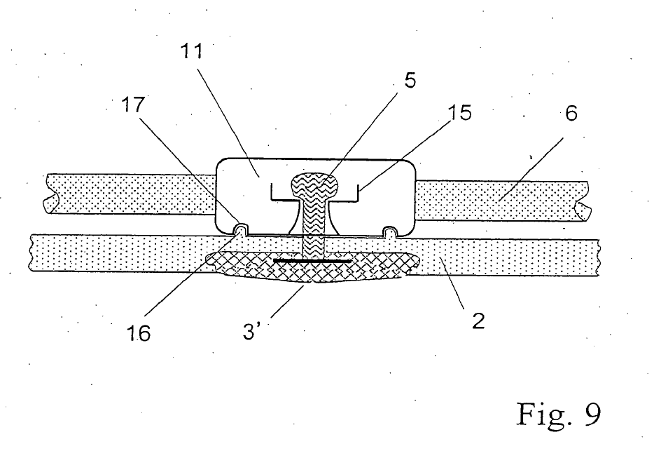

FIG. 9 shows a schematic view of a connection element 11 with an electrode attached thereto, wherein the area of the electric contact is secured against the penetration of liquids. A sealing bead 16 is made integrally in one piece with the elastic belt 2 in the area of contact with the connection element 11. The body of the connection element 11 has a groove 17 which is complementary to the sealing bead 16. The sealing bead engages the groove 17 in a positive-locking manner. Nevertheless, the connection remains rotatable. All advantageous embodiments of the present invention can thus be utilized combined with an especially secure contacting.

While specific embodiments of the invention have been shown and described in detail to illustrate the application of the principles of the invention, it will be understood that the invention may be embodied otherwise without departing from such principles.

What is claimed is:

1. An electrode belt for carrying out electrodiagnostic procedures on the human body, the electrode belt comprising:
a plurality of connection elements;
a multicore cable, said plurality of connection elements being positioned along said multicore cable, one connection element being located at a spaced location from another connection element, each connection element being connected to a lead of said multicore cable;
a belt formed at least partially of an elastic material and having a length for surrounding the body of a test subject, said belt being movable from a relaxed state to a stretched state, said belt having a first length in said relaxed state, said belt having a second length in said stretched state, said second length being greater than said first length;
a plurality of electrodes connected to the belt for being in flat contact with the body of the test subject, each electrode being positioned along said belt at a spaced location from an adjacent electrode, said elastic material being provided between adjacent electrodes with each electrode moving relative to said adjacent electrode when said belt moves from said relaxed state to said stretched state, whereby said electrodes carry out an electroimpedance tomography procedure in said stretched state; and
a plurality of contact means each for mechanically and electrically connecting and mechanically and electrically disconnecting from a respective one of said connection elements, each contact means extending from a respective one of said electrodes through said belt to a position located outside of said belt, each of said connection elements having a retaining means for mechanically and electrically connecting to one of said contact means, said retaining means being flexible to generate a snap in retaining function as said contact means is inserted into said retaining means, whereby each contact means is mechanically and electrically connected to said multicore cable via said retaining means.

2. An electrode belt in accordance with claim 1, wherein said multicore cable has a multicore cable length, said multicore cable length being thirty percent greater than said first length.

3. An electrode belt in accordance with claim 1, wherein said multicore cable has a plurality of multicore cable portions, each multicore cable portion extending between one of said connection elements and an adjacent connection element, each multicore cable portion having a multicore cable portion length, each of said electrodes and an adjacent electrode defining a first distance between electrodes in said relaxed state, said first distance between electrodes being less than said multicore cable portion length.

4. An electrode belt in accordance with claim 3, wherein one or more of said multicore cable portions extends in a meandering pattern when said belt is in said relaxed state, one or more of said multicore cable portions extending substantially parallel to said belt when said belt is in said stretched state.

5. An electrode belt in accordance with claim 1, wherein at least one of said connection elements rotates about a longitudinal axis of one of said contact means when said belt moves from said relaxed state to said stretched state.

6. An electrode belt in accordance with claim 3, wherein each of said electrodes and an adjacent electrode define a second distance between electrodes in said stretched state, said second distance being greater than said first distance.

7. An electrode belt in accordance with claim 1, wherein each contact means and an adjacent contact means define a first contact means spacing when said belt is in said relaxed state, each contact means and an adjacent contact means defining a second contact means spacing in said stretched state, said second contact means spacing being greater than said first contact means spacing.

8. An electrode belt in accordance with claim 1, wherein each of said contact means comprises a rigid contact pin connected to an electrode, the rigid contact pin passes through said belt, protrudes from said belt on the side of said belt facing away from the body, and is detachably connected to one of said corresponding connection elements.

9. An electrode belt for electrical impedance tomography, the electrode belt comprising:
an electrode holding belt structure formed at least partially of an elastic material and having a length for surrounding the body of a test subject, said electrode holding belt structure being movable between a non-stretched state and a stretched state;
a plurality of electrodes integrated with and fixed to said electrode holding belt structure to provide flat or convex contact with a surface of the test subject, said elastic material being arranged between one of said electrodes and another one of said electrodes;
a multiline cable with a plurality of electrode feed lines;
a plurality of connection elements, each of said connection elements being connected to one of said electrode feed lines and fixed to said multiline cable at spaced locations along said multiline cable providing spacing between adjacent connection elements; and
a plurality of contacts, each leading from a respective one of said electrodes, each contact having a size and shape, each of said plurality of connection elements having a spring contact for mechanically and electrically connecting to a respective one of said plurality of contacts and for mechanically and electrically disconnecting from said respective one of said plurality of contacts, said spring contact having spaced apart ends defining a contact insertion gap that is smaller than said size of said contact to generate a snap in retaining function when said contact is connected to said connection element with said spring contact in electrical contact with a respective said contact, each of said electrodes moving relative to an adjacent electrode when said electrode holding belt structure moves from said non-stretched state to said stretched state, each of said contacts moving relative to an adjacent contact when said electrode holding belt structure moves from said non-stretched state to said stretched state, each of said connection elements moving relative to an adjacent connection element when said electrode holding belt structure moves from said non-stretched state to said stretched state.

10. An electrode belt in accordance with claim 9, wherein said electrode holding belt structure has a first length in said non-stretched state, said belt having a second length in said stretched state, said second length being greater than said first length.

11. An electrode belt in accordance with claim 9, wherein each of said connection elements and an adjacent connection element define a first connection element space when said electrode holding belt structure is in said non-stretched state, each of said connection elements and an adjacent connection element defining a second connection element space when said electrode holding belt structure is in said stretched state, said second connection element space being of a dimension that is greater than a dimension of said first connection element space.

12. An electrode belt in accordance with claim 10, wherein said multiline cable has a multiline cable length, said multiline cable length being thirty percent greater than said first length.

13. An electrode belt in accordance with claim 9, wherein said multiline cable has a plurality of multiline cable portions, each multicore cable portion extending between one of said connection elements and an adjacent connection element, each multiline cable portion having a multiline cable portion length, each of said electrodes and an adjacent electrode defining a first distance between electrodes in said non-stretched state, said first distance between electrodes being less than said multiline cable portion length.

14. An electrode belt in accordance with claim 13, wherein one or more of said multiline cable portions extends in a meandering pattern when said electrode holding belt structure is in said non-stretched state, one or more of said multiline cable portions extending substantially parallel to said electrode holding belt structure when said electrode holding belt structure is in said stretched state.

15. An electrode belt in accordance with claim 9, wherein each of said connection elements is rotatably connected to one of said contacts, at least one of said connection elements rotating about a longitudinal axis of one of said contacts when said electrode holding belt structure moves from said non-stretched state to said stretched state.

16. An electrode belt in accordance with claim 9, wherein each of said contacts and an adjacent contact define a first contact spacing when said electrode holding belt structure is in said non-stretched state, each of said contacts and an adjacent contact defining a second contact spacing when said electrode holding belt structure is in said stretched state, said second contact means spacing being greater than said first contact means spacing.

17. An electrode belt in accordance with claim 9, wherein each of said contacts comprises a rigid contact pin connected to one of said electrodes, said rigid contact pin passes through said electrode holding belt structure, protrudes from said electrode holding belt structure on the side of said belt facing away from the body, and is detachably connected to one of said connection elements.

18. An electrode belt in accordance with claim 13, wherein one or more of said multiline cable portions extends in a meandering pattern when said electrode holding belt structure is in said non-stretched state, one or more of said multicore cable portions extending substantially parallel to said electrode holding belt structure when said electrode holding belt structure is in said stretched state.

19. An electrode belt in accordance with claim 9, wherein said electrodes carry out an electroimpedance tomography procedure in said stretched state.

20. An electrode belt for electrical impedance tomography, the electrode belt comprising:
a belt structure having a plurality of electrodes connected therein, one electrode being located along said belt structure at a spaced location from another electrode to define an electrode spacing, each electrode having a contact extending therefrom, said holding belt structure being formed at least partially of an elastic material and having a length for surrounding the body of a patient, said belt structure being movable from an unapplied state to an applied state, said elastic material being located between each electrode and an adjacent electrode, said belt structure not being stretched in said unapplied state, said belt structure being stretched in said applied state, each electrode moving relative to an adjacent electrode in said applied state, said electrode spacing having a first dimension in said unapplied state, said electrode spacing having a second dimension in said applied state, said second dimension being greater than said first dimension, said electrodes performing an electroimpedance tomography procedure in said applied state;
a multicore cable having a plurality of connection elements, one connection element being located along said multicore cable at a spaced location from another connection element;
a snap retaining means associated with one of said contacts and one of said connection elements, whereby each said connection element is connected to a respective said contact via said snap retaining means after said belt structure is applied to the patient, said snap retaining means mechanically and electrically connecting each said contact to said multicore cable.
